(19) United States
(12) Patent Application Publication
(10) Pub. No.: US 2025/0176909 A1
(43) Pub. Date: Jun. 5, 2025

(54) APPARATUS FOR MEASURING VITAL PARAMETERS

(71) Applicant: FRESENIUS MEDICAL CARE DEUTSCHLAND GMBH, Bad Homburg (DE)

(72) Inventor: Gerome FISCHER, Bad Homburg (DE)

(73) Assignee: FRESENIUS MEDICAL CARE DEUTSCHLAND GMBH, Bad Homburg (DE)

(21) Appl. No.: 18/704,067

(22) PCT Filed: Oct. 28, 2022
(86) PCT No.: PCT/EP2022/080171

§ 371 Date: Apr. 24, 2024

(30) Foreign Application Priority Data
Oct. 28, 2021 (DE) 10 2021 212 183.7

(51) Int. Cl. A61B 5/00

(52) U.S. Cl. CPC A61B 5/6804; A61B 2562/04; A61B 2562/12; A61B 2562/164; A61B 2562/166

(57) ABSTRACT

The invention relates to device for the measurement of vital parameters, wherein the device is suitable for wearing on the skin, and wherein the device comprises:
an elastic carrier (W), and
a plurality of sensors (S1 . . . N), wherein the sensors (S1 . . . N) are geometrically extended and identical in terms of their shape,
wherein at least two sensors from the plurality of sensors (S1 . . . N), are of different sizes in comparison with each other, wherein
the plurality of sensors (S1 . . . N) are printed onto to the elastically carrier (W).

The invention relates to device for the measurement of vital parameters.

### BACKGROUND

In many fields, the recording of vital data is desirable. Examples of the most prominent fields are sport and (human) medicine. Vital data, such as respiratory frequency or heart rate/pulse, are important parameters with which the strain on a body can be recognised.

However, up until now, devices have been comparatively expensive and are not reliable when recording parameters. Sensors with which such vital parameters can be recorded, must be carefully positioned. In order to prevent slipping out of place, e.g. during (sporting) activity, such known sensor are in close contact with the skin. Frequently this results in bruising and also skin irritation. Sweat can also build up. Through this there is then also the risk of the skin being target of fungal infestation.

In the recent past, so-called smart textiles have been proposed. However, these also have the problem of poor reliability, particularly during movement. Furthermore, with these so-called smart textiles, the problem constantly arises that these are only intended for one user size and careful use. However, if, as to be expected in the case of movement, stretching of the material occurs, these smart textiles could no longer reliably measure a desired vital parameter.

Equally, reproducibility is a problem if the measuring location changes each time they are newly applied. This is, however, to be expected with the comparatively small sensors.

This means, that when applying such measuring devices, there is the problem of applying the measuring sensors in such a way that a sensor is placed at a measuring location that is well suited for measurement. In the case of repeated application, there is also the problem of reproducibly finding an adequate measuring location. And thirdly, with geometrically expanded sensors there is the problem that, through application, the sensor structure can be stretched or compressed so much that it loses sensitivity for the desired measuring signal. During application, the sensor structures stretch with the expansion of the elastic carrier material. This expansion can differ in individual application procedures. Through this, the further problem of reproducibility occurs again.

In summary, with known wearables there is the problem that through variations during application, a useable measuring signal cannot be reliably obtained.

### Objective

On the basis of this, an objective of the invention is to provide a device that is suitable for different users and also allow reliable measurement even during movement.

### BRIEF DESCRIPTION OF THE INVENTION

This objective is achieved by the device according to the invention for the measurement of vital parameters, wherein the device is suitable for wearing on the skin and wherein the device comprises an elastic carrier and a plurality of sensors, the sensors being geometrically expanded and identical in terms of their shape, wherein the plurality of sensors are applied to, for example, printed onto the elastic carrier.

In one embodiment of the invention, the elastic carrier comprises a textile material.

In accordance with a further embodiment of the invention, the device also comprises a further spatially concentrated sensor.

In yet another embodiment of the invention, the plurality of sensors has finger-like sections.

According to yet another embodiment of the invention, the plurality of sensors has stepped, meandering or spiral-like sections.

In a further embodiment of the invention, the plurality of sensors is set up to record a vital parameter.

According to another embodiment of the invention, from the plurality of sensors in operation, the device selects at least one sensor, which, in comparison with other sensors in the plurality of sensors, provides the greatest signal amplitude during a measuring period.

In yet a further embodiment of the invention, from the plurality of sensors in operation, the device brings together at least one sub-group of sensors for a common signal output.

In yet a further embodiment of the invention, each of the sensor from the plurality of sensor in operation is read out individually.

In a further embodiment of the invention, the plurality of sensors is imprinted on the elastic carrier by means of screen printing in one or more layers.

According to a further embodiment of the invention, the plurality of sensors has intervals between the structures of at least 15 μm.

In yet another embodiment of the invention, at least two sensors of the plurality of sensors are of different sizes compared with each other.

The objective is also achieved by the use according to the invention of a device according to the invention.

In accordance with another embodiment of the invention, a device according to the invention is used to monitor a patient or a sportsperson.

Further advantageous embodiments are the subject matter of the respective dependent claims, the figures and the description.

### BRIEF DESCRIPTION OF THE FIGURES

The invention will be described in more detail below by way of a drawing and examples of embodiment. The drawing is a schematic view and is not true to scale. The drawing does not limit the invention in any way.

Here:

FIGS. 1 and 2 show a schematic view of geometrically expanded sensors that are identical in shape according to forms of embodiment of the invention.

FIGS. 3 and 4 show a schematic view of further geometrically expanded sensors that are identical in shape according to forms of embodiment of the invention.

FIGS. 5 and 6 show a schematic view of yet further geometrically expanded sensors that are identical in shape according to forms of embodiment of the invention.

FIG. 7 shows a schematic view of sensor sections that can form one or more sensors according to forms of embodiment of the invention, and

FIG. 8 shows a schematic view of a device in accordance with forms of embodiment of the invention.

### DETAILED DESCRIPTION OF THE INVENTION

The invention will be described in more detail below with reference to the figures. It should be noted that different aspects are described which can each be used individually or in combination. This means that each aspect can be used with different forms of embodiment of the invention unless explicitly set out as a pure alternative.

Furthermore, for the sake of simplicity reference will generally only be made to one entity. However, unless specifically stated, the invention can also comprise several of the entities in question. To this end, the use of the words “a” and “an” should only be taken as an indication that in a simple form of embodiment at least one entity is used.

Insofar as methods are described below, the individual steps of a method can be arranged and/or combined in any order unless otherwise explicitly evident from the context. In addition, the methods-unless expressly characterised differently—can be combined with each other.

As a rule, indicated numerical values should not be understood as exact values, but include a tolerance of +/−1% to +/−10%.

Insofar as standards, specifications or similar documents are cited in this application, reference is always made to the standards, specifications or similar documents applicable on the date of application. This means that if a standard/specification etc. is updated or replaced by a successor, the invention is also applicable to these.

In the figures, various forms of embodiment are shown. In particular, in FIG. 8, a device for the measurement of vital parameters is schematically shown as an example.

The device is suitable for wearing on the skin. That is to say, when used as intended, the device comprises a skin-friendly, biocompatible material on the side directed towards the body.

More particularly, the device has an elastic carrier W. This elastic carrier W can—as shown schematically in the figure—be designed in a hose-like manner so that it can be worn elastically around, for instance, an extremity (e.g. upper or lower arm) or around the upper body (e.g. chest, torso). It can also be designed as a strap which, by means of a Velcro closure for example, can be connected in a hose-like manner and also applied around an extremity or around the upper body. In particular, the carrier W is breathable. The term elastically not only covers deformability, but also allows expansion. For example, an expansion of the diameter d by up to 5% or more, e.g. up to 10%, or up to 15%, or up to 20%, or up to 25% can be provided, depending on carrier material used and/or the material for the sensors. On the one hand, elasticity allows the device to be applied to the body and on the other hand it provides comfort when wearing. Moreover, through the elasticity, good contact can also be achieved for carrying out measurements (relative) to the skin/the body of the wearer.

In general, the device according to the invention can therefore also be designated as a wearable.

The device also comprises a plurality of sensors S_{1 . . . N}, wherein the sensors S_{1 . . . N} are geometrically expanded and identical in terms of their shape. Obviously the S_{1 . . . N} can be geometrically distributed, i.e. the sensors take up differently sized areas and have, at least in parts, different conducting sections. However, without loss of generality, various sensors can also comprise overlapping areas or and/or have common conducting areas. Spatial overlapping/co-use of conducting sections can be used for the local preference of one sensor in relation to an area to be measured. By contrast, spatial separation can be used in order to be able to investigate different spatial areas.

In particular, geometrically expanded is to be understood as different from more punctiform sensors S_{T}. Punctiform sensors can, for example, be temperature sensors, such as a PT100 sensor. These are concentrated locally. Geometrically expanded sensors S_{1 . . . N} have, for example, the property that they comprise conductor-like sections which through expansion or through the change of distance between (conductor-like) sections alter at least one electrical parameter, on the basis of which measurements of vital parameters can be carried out.

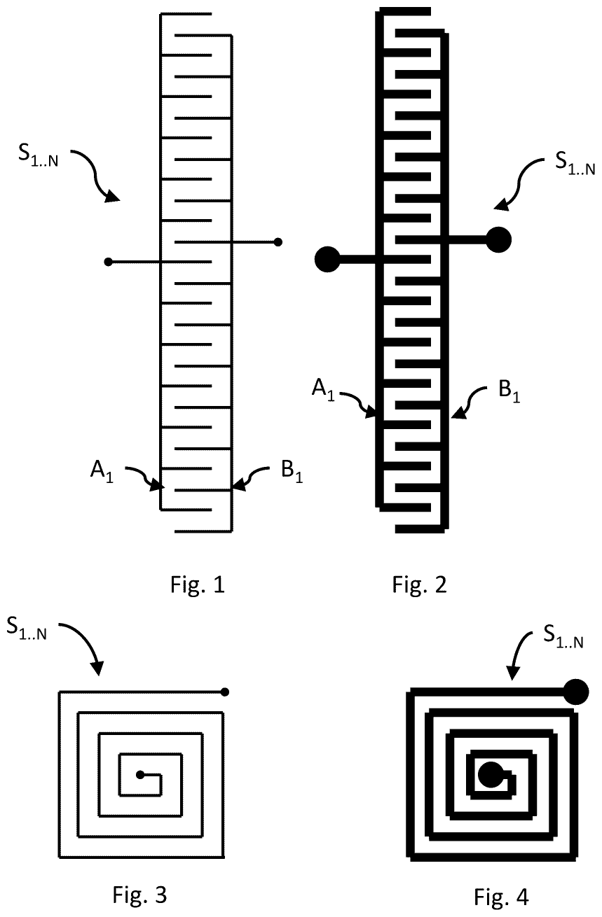

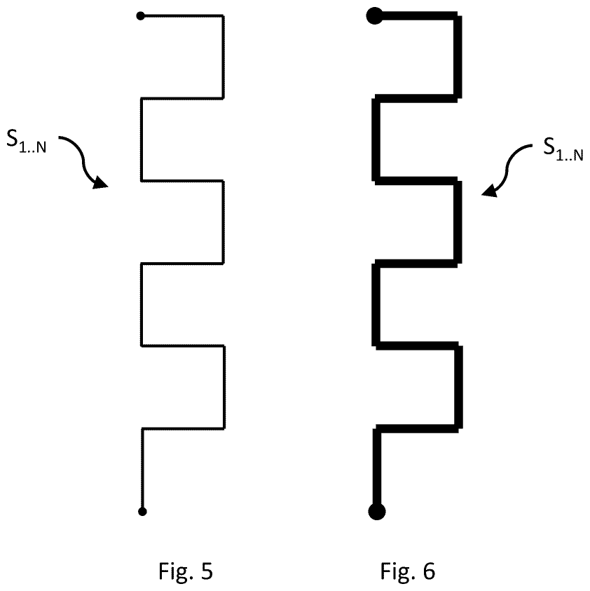

Here, in forms of embodiment in the delivery condition, at least two sensors of the plurality of sensors S_{1 . . . N} are already of different sizes compared with each other. For example, in a comparison of FIG. 1 with FIG. 2 or FIG. 3 with FIG. 4 or FIG. 5 with FIG. 6, the conductor sections have a different conductor width. In addition, in a comparison of FIG. 1 with FIG. 2, the conductor sections A1 to B1 are at a different distance from one another. In a comparison of FIG. 3 with FIG. 4 it can also be seen that the distance between the conductor sections in the interior in FIG. 4 is smaller than in FIG. 3. Without loss of generality, it can as a rule be said the basic geometrical shape can be similar, but the size, for example, can differ (proportionally).

In accordance with the forms of embodiment, the plurality of sensors S_{1 . . . N} can be applied to, e.g. printed onto (e.g. by means of screen printing), woven into or adhered to, the elastic carrier W. In this respect, printing, in particular screen printing, is at the present time an especially cost-effective option for applying sensors to an elastic carrier W.

As the sensors S_{1 . . . N} or the conductive sections constituting them, as well as the distances between them, are subject to expansion in a device worn in this way, the change caused by the expansion can be used as a measurement variable. If the device is worn around the chest, for example, the respiratory frequency can be determined through the expansion of the chest.

Here, it can be established that when using the sensors, the strength of a signal amplitude (particularly in a voltage signal amplitude differing from 0 V) of a respective sensor S_{1 . . . N} is dependent on the current expansion (e.g. expansion through wearing on the body). In this way, a distinction can be made between a device that is not worn and a device that is worn. However, this property also means that depending on the respective expansion due to wearing, each sensor S_{1 . . . N} can be (differently) expanded. Different expansion of the sensors S_{1 . . . N} thus also provides a possibility of intrinsic filtering.

More especially, the elastic carrier W can comprise a textile material. This provides, in particular, elasticity and also breathability, and therefore also wearing comfort.

In one form of embodiment of the invention, the device also comprises a further spatially concentrated sensor S_{T}. This means that the device can, for example, in addition to sensors which make a measuring signal available on the basis of expansion, comprise further sensors, for instance a temperature sensor.

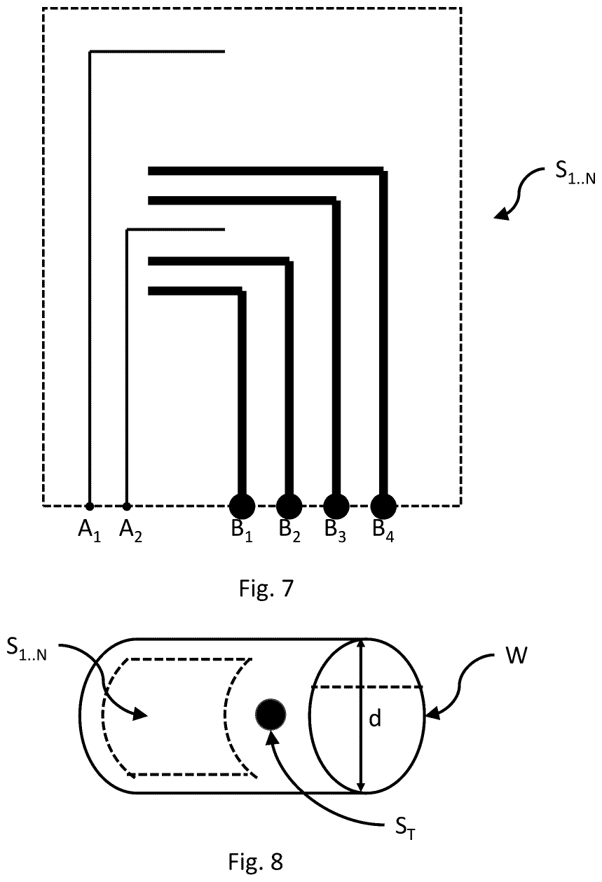

According to one form of embodiment of the invention, the plurality of sensors S_{1 . . . N} comprises finger-like (interdigital) sections as shown in FIG. 1 or 2. With such structures, capacity-sensitive sensors, for example, can be constructed. If the distances between the finger-like sections A₁ and B₁ change, the capacity of the sensors S_{1 . . . N} is altered. An identical situation can be seen in FIG. 7 between the finger-like sections A_{1 . . . 2} und B_{1 . . . 4}. This means that the distances between adjacent “fingers” can be the same or varied. Through this the sensitivity of the structures varies, but the measuring principle remains the same. However, the sensor structures are mathematically similar. The sensitivity for a particular measuring range arises from a particular expansion at which measuring signals for effects are especially strong.

In accordance with a further form of embodiment, the plurality of sensors S_{1 . . . N} has stepped, meandering or spiral-like sections. For example, in FIGS. 3 and 4, spiral-like sections are shown, whereas FIGS. 5 and 6 show meandering sections. Sections A₁, A₂, B_{1 . . . 4} in FIG. 7 can each be considered as a step.

In the embodiments of the invention, the plurality of sensors S_{1 . . . N} is set up to record a vital parameter. An example of a vital parameter is the respiratory frequency, which can, for instance, be determined when the device surrounds the chest. Another vital parameter can be the heart rate for example. The pulse/heart rate can, for example, be determined through the structures of the device. Through changes in the course of the signal, conclusions can be drawn about stenoses and thromboses. By way of temperature measurements, conclusions can also be drawn about an existing infection. Temperature measurements on the spatially extended sensors S_{1 . . . N} are possible as, governed by the conductor material used, the conductivity is temperature-dependent. Other vital parameters can, for example, be the conductivity of the skin if a sensor S_{1 . . . N} is in contact with the skin at different spatially separated points. Through this the moisture on the skin can be determined, for example.

Further vital parameters can be other detailed properties of these vital functions, for example the depth of breaths (e.g. measurement of the capacity), intensity of the heartbeats or also the relative intensity of the various components of a heartbeat (two chambers in relation to each other), skin tonus (i.e. goosebumps) and, in the case of wearing around the arm, the chest or the leg, also collective movement events such as a muscle contraction. A distinction can also be made between a contracted and relaxed muscle and, if necessary, a baseline established in order to determine whether the muscle in question is indeed (fully) relaxed.

In a further development, the measuring signal of all sensors S_{1 . . . N} of a device according to the invention are read out together (i.e. in one channels so to speak). Here, on the one hand it is especially advantageous that technically particularly simple and therefore particularly cost-effective evaluation electronics, i.e. with only one channel, can be used. On the other hand it is especially advantageous that the measuring signal of the sensor S_{1 . . . N} applied at the best measuring location and/or with the best geometrical extension, will dominate the entire measuring field of the other sensors S_{1 . . . N} as its signals is the strongest. This also applies in particular in the case of resonant measuring principles.

Furthermore, physiological events, which are measured in the form of vital parameters, are often perceived collectively by all sensors S_{1 . . . N} practically simultaneously. A breath, a muscle contraction or a heartbeat occur on time scales that are orders of magnitude greater than the duration of the measuring signals, so that they are measurable practically at the same time for all sensors S_{1 . . . N}. Therefore, through simultaneous measurement by all sensors S_{1 . . . N} a vital parameter can be expediently measured. This means that it is also not disadvantageous if there are several sensors S_{1 . . . N} with similar signal quality.

To a certain extent, the device allows “intrinsic filtering” to the dominant sensors S_{1 . . . N} Even in the event of a dynamic change, e.g. a movement by the wearer, during breathing or the course of a pulse, there is no problem, as—if expansion or locations of sensors S_{1 . . . N} change—another sensor S_{1 . . . N} can now provide a better signal quality and thereby the measuring signals of all sensors S_{1 . . . N} of a device according to the invention can continue to provide a good evaluable signal. In the case of small area expansions (e.g. less than 5 mm², but more particularly less than or equal to 1 mm²) of the sensors S_{1 . . . N}, signals to be measured physiologically, such as those brought about by heartbeats, do not normally result in the dominant sensor S_{1 . . . N} changing.

According to another form of embodiment of the invention, from the plurality of sensors S_{1 . . . N} in operation, the device selects at least one sensor S_{1 . . . N}, which, in comparison with other sensors in the plurality of sensors S_{1 . . . N}, provides the greatest signal amplitude during a measuring period. For example, if in FIG. 7, in an assumed case, the sensor S_{1 . . . N} formed of conductor sections A₁ and B₃ delivers the greatest signal amplitude in a measuring period, this section combination is preferred to other section combinations, such as, for example, A₂ und B₁. This allows a particularly suitable signal to be found selectively. This is of particular advantage if the signal is to be further processed in high resolution in order, for example, to provide further evaluations.

However, for this, it can also be envisaged that from the plurality of sensors S_{1 . . . N} in operation, the device combines at least one sub-group of sensors S_{1 . . . N} for a joint signal output. For example, it could be envisaged that is combines the conductor sections A₁ and B₄ as well as also the conductor sections A₂ and B₁ for a joint signal output (i.e. section A₁ and A₂ or section B₁ and B₄). As an example, in each case 10 out of N=100 sensors S_{1 . . . N} can be jointly read out in one channel of a measuring electronics system. Here too there is the particular cost advantage of requiring fewer channels than if each sensor were to be read out individually, as well as the “intrinsic filtering” by way of the plurality of sensors.

However, there is the additional benefit that various groups of sensors S_{1 . . . N} can also be differentially evaluated vis-a-vis each other and further findings obtained in this way.

If can, of course, also be envisaged that each of the sensors S_{1 . . . N} from the plurality of sensors S_{1 . . . N} in operation is individually read out. For example, in FIG. 7 each of the sensors S_{1 . . . N} formed of a pair of conductor sections A₁, A₂ and B_{1 . . . 4} can be considered as a single sensor S_{1 . . . N} that can each be individually read out. Here, individually does not necessarily means separated in terms of time, as in FIG. 7 it would, for example, be possible to read out the pair A₁/B₄ (as one sensor S_{1 . . . N}) and the pair A₂ and B₁ (as a further sensor S_{1 . . . N}) at the same time. This means that in this further development every sensor S_{1 . . . N} can be read out individually in a separate channel of an evaluation electronics system. Particularly advantageously, the data evaluation could be especially precise in that only particularly strong signals (of especially well placed/expanded sensors S_{1 . . . N}) are specifically considered but also evaluated vis-a-vis each other.

Of course, the use of mixed forms of the forms shown above as alternatives can also be envisaged. This could be brought about through temporally sequential wiring, for example. As a result of this, vital parameters can be determined in different mays and with different degrees of detail. In one form of embodiment of the invention, the plurality of sensors S_{1 . . . N} is printed onto the elastic carrier W by means of screen printing. Screen printing is a cost-effective process which allows identical sensors S_{1 . . . N} to be applied to a plurality of differently shaped carriers W.

According to yet another form of embodiment of the invention, the plurality of sensors S_{1 . . . N} is printed onto the elastic carrier W in several layers by means of screen printing. Multiple layer printing allows the creation of complex sensors S_{1 . . . N}, but can also be used to increase the conductivity, for example. Through this, lead resistance and flexibility can be optimally matched to the purpose of use.

According to a further embodiment of the invention, the plurality of sensors S_{1 . . . N} has spacings between the structures of at least 15 μm. Through this, sensors S_{1 . . . N} can be produced which on the one hand are sensitive, but on the other hand are also mechanically stable.

By way of the present invention it is possible to provide a plurality of geometrically similar, geometrically differently expanded sensors S_{1 . . . N} (e.g. interdigital structures, spiral structures, labyrinth patterns etc.).

Through this, a “self-correcting” wearable sensor S_{1 . . . N} for vital parameters can be provided. Among other reasons, this is possible in that in a plurality of such sensors S_{1 . . . N} there is an increased probability of at least one sensor S_{1 . . . N} providing a desired signal/a desired signal amplitude. That is to say, that the probability increases that in the case of at least one of the sensors S_{1 . . . N} in the plurality of sensors S_{1 . . . N} operation/measurement can take place (close enough) to a working point of the measurement/resonance case.

Although in principle it is possible to also provide evaluation electronics for the sensors S_{1 . . . N} on the carrier W (as well as a power supply), these can also be separate from the carrier W and only be connected to the sensors S_{1 . . . N} on the carrier W when required. For this, suitable wired or wireless interfaces can be provided. For example, it can be envisaged that energy can be obtained from an alternating field by means of coils on the carrier W. Likewise, via an antenna, a (pre)-processed (digital) measuring signal can be made available, e.g. by way of Bluetooth, Bluetooth-LowEnergy, Wifi, DECT, DECT-ULE or another near-field communications technology. Equally, for instance, a USB interface or similar can be provided. Such interfaces can also be integrated into a Velcro closure. Advantageously, such an interface is small and arranged on the side facing away from the body so that the wearing comfort does not suffer. However, in principle it is also possible to provide a (rechargeable) battery structure on the carrier by way of screen printing.

The sensor data can be both locally (pre)-processed as well as also (post)-processed on a remote device, e.g. a smartphone, a workplace computer.

Preferably, the device is worn directly on or as close as possible to the body. However, in the case of certain measurements, for example, respiratory frequency, it can also be worn on clothing.

Without loss of generality, it can be envisaged that in periods during which no measurements by the conductor sections as part of a sensor S_{1 . . . N} are taking place, the conductor section can also be used for the (continuous or pulsed) conversion of electrical energy into heat energy.

The conducting sections of the sensors S_{1 . . . N} can be made of a variety of possible materials and comprise, for example, silver, gold, carbon, polyaniline etc.

If the measuring signals contain much noise, the processing of period signal, such as respiratory frequency or heart rate, can involve processing by way of a Fourier transform. Here, (analogue) prefiltering (e.g. on the device) through passive (high/low/band pass filter, band block) as well as digital filtering can of course be alternatively or additionally used.

Also, through suitable selection of conductor sections or their shaping, for example, the coupling/detection of interference signals can be avoided or at least kept low.

Without loss of generality, the device can be designed both as a disposable article as well as a reusable product. It is particularly preferable that the device is hygienically sterilisable so that it can be used as a medical product. Preferably the device is also washable at high temperatures (75° C.-95°).

For example, the device can be used for monitoring dialysis patients. When used in this way, the device is worn directly on the fistula/graft, therefore mainly on the lower arm or the arm. This can be designed as a disposable article (disposable), for multiple use (durable) or as a permanent wearable.

The invention makes it possible for a plurality of printed-on sensors S_{1 . . . N} to be provided. When applying the device, a sufficient number of sensors S_{1 . . . N} contact suitable locations for measurement and undergo an extension that is suitable for measuring.

It should be noted, that for reproducibility, the same sensors S_{1 . . . N} and the same expansion do not always have to be taken into account. Both with regard to the “suitable measuring location” and also the “suitable geometrical expansion” of a sensor S_{1 . . . N}, such a solution manages without further aids and therefore makes simple, inherently failsafe handing possible.

In the context of the invention, it is possible to print several layers by means of screen printing. This can be 2 to 4 layers for example. In this way, particularly advantageously, more efficient possibilities of connecting the sensor structures or more complex sensor structures are made possible.

The devices according to the invention can thus be used in particular for the monitoring of a patient, e.g. a dialysis patient, but also for monitoring a sportsperson.

1. A device for the measurement of vital parameters, wherein the device is suitable for wearing on the skin, and wherein the device comprises:
an elastic carrier, and
a plurality of sensors, wherein the sensors are geometrically expanded and identical in terms of their shape,
wherein the plurality of sensors is applied to the elastic carrier.

2. The device according to claim 1, wherein the elastic carrier comprises a textile material.

3. The device according to claim 1, wherein the device also comprises a further spatially concentrated sensor.

4. The device according to claim 1, wherein the plurality of sensors has finger-like sections.

5. The device according to claim 1, wherein the plurality of sensors comprises stepped, meandering or spiral-like sections.

6. The device according to claim 1, wherein the plurality of sensors is set up to record a vital parameter.

7. The device according to claim 1, wherein from the plurality of sensors in operation, the device selects at least one sensor, which in comparison with other sensors in the plurality of sensors, provides the greatest signal amplitude during a measuring period.

8. The device according to claim 1, wherein from the plurality of sensors in operation, the device selects at least one sub-group of sensors for a joint signal output.

9. The device according to claim 1, wherein each of the sensors from the plurality of sensors in operation is individually read out.

10. The device according to claim 1, wherein the plurality of sensors is printed onto the elastic carrier by means of screen printing.

11. The device according to claim 1, wherein the plurality of sensors is printed onto the elastic carrier in several layers by means of screen printing.

12. The device according to claim 1, wherein the plurality of sensors has a structure spacing of at least 15 μm.

13. The device according to claim 1, wherein at least two sensors from the plurality of sensors are of different sizes in comparison with each other.

14. The device according to claim 1, wherein the plurality of sensors is printed onto the elastic carrier.

15. A method for monitoring a patient, said method comprising utilizing the device of claim 1.

16. A method for monitoring a sportsperson, said method comprising utilizing the device of claim 1.
